(19) United States
(12) Patent Application Publication
(10) Pub. No.: US 2015/0150843 A1
(43) Pub. Date: Jun. 4, 2015

(54) CARDIOPROTECTIVE COMPOUNDS, THEIR USE WITH CHEMOTHERAPY, AND METHODS FOR IDENTIFYING THEM

(71) Applicants: Randall T. Peterson, Belmont, MA (US); Yan Liu, Burlington, MA (US)

(72) Inventors: Randall T. Peterson, Belmont, MA (US); Yan Liu, Burlington, MA (US)

(21) Appl. No.: 14/402,512

(22) PCT Filed: May 16, 2013
(86) PCT No.: PCT/US2013/041334

§ 371 Date: Nov. 20, 2014

Related U.S. Application Data
(60) Provisional application No. 61/780,338, filed on Mar. 13, 2013.
(60) Provisional application No. 61/649,626, filed on May 21, 2012.

(51) Int. Cl. A61K 31/352; A61K 49/00

(52) U.S. Cl. CPC A61K 31/352; A61K 49/0008

(57) ABSTRACT

The invention provides a method of reducing anthracycline-induced cardiotoxicity by administering a toxicity-reducing compound, such as a compound of Formulas (I), (II) or (III) or a combination thereof, and/or diphenyl urea (DPU), dexrazoxane (DEX), to a patient receiving an anthracycline. The invention also provides a method of identifying toxicity-reducing compounds.

### RELATED APPLICATIONS

This application claims the benefit of priority to U.S. Provisional Patent Application No. 61/780,338, filed Mar. 13, 2013 and U.S. Provisional Patent Application No. 61/649,626, filed May 21, 2012, which applications are hereby incorporated by reference in their entirety.

### STATEMENT REGARDING FEDERALLY SPONSORED RESEARCH OR DEVELOPMENT

This invention was supported in part by the United States Government under National Heart, Lung and Blood Institute Grant T32HL007208. The Government may have certain rights in this invention.

### BACKGROUND OF THE INVENTION

Doxorubicin is a very potent chemotherapy drug widely used against a broad range of cancers including solid tumors, soft tissue tumors and leukemia (Weiss et al., Cancer Chemother. Pharmacol. 18, 185-197, 1986). Like other members of the anthracycline class, its usage is greatly limited due to its cardiotoxicity. Cumulative dosages above 450 mg/m² exponentially increase the risk of heart failure. Even at lower doses, a number of patients still inevitably develop heart disease in later years (Chen et al., Circ. Res. 108, 619-628, 2011). The underlying mechanisms of this cardiotoxicity have not been fully understood. Nevertheless, increased reactive oxygen species (ROS) production and cardiomyocyte apoptosis have been widely documented to play critical roles (Chen et al., Circ. Res. 108, 619-628, 2011). Of note, doxorubicin has high affinity to cardiolipin, a component of the mitochondrial inner membrane (Goormaghtigh et al., Biophys. Chem. 35, 247-257, 1990). This, coupled with the fact that cardiomyocytes have the highest mitochondrial content among all cell types (Barth et al., J. Mol. Cell Cardiol. 24, 669-681, 1992) may explain why the heart is among the organs most sensitive to doxorubicin toxicity. Doxorubicin induces apoptosis in cardiomyocytes and tumor cells via distinct and overlapping mechanisms. Traditionally, doxorubicin is regarded as a topoisomerase II inhibitor, causing double-stranded DNA breaks and consequently triggering p53 dependent apoptosis in neoplastic cells5. In cardiomyocytes, doxorubicin induces cell death primarily by promoting ROS production (Horenstein et al., Mol. Genet. Metab. 71, 436-444, 2000). Recently, the role of ROS in doxorubicin induced tumor cell death has become more widely appreciated, especially in tumors bearing p53 mutations (Fang et al., J. Drug. Target, 15, 475-486, 2007; Das et al., Proc. Natl. Acad. Sci. USA, 107, 18202-18207, 2005). On the other hand, the role of p53 in cardiomyocyte apoptosis remains controversial. Although p53 deletion or inhibition provides protection against doxorubicin induced heart failure (Shizukuda et al., Mol. Cell Biochem, 273, 25-32, 2005; Liu et al., Am. J. Physiol. Heart Circ. Physiol. 286, H933-H939, 2004), direct evidence supporting a role for p53 in doxorubicin induced cardiomyocyte apoptosis is lacking (Feridooni et al., PLoS. One 6, e22801, 2011). So far, dexrazoxane is the only approved drug clinically used to ameliorate doxorubicin-induced heart failure in chemotherapy. It is believed to chelate intracellular iron and block iron assisted radical production (Hasinoff et al., Cardiovasc. Toxicol. 7, 140-144, 2007). Nevertheless, its usage is limited because of concerns that dexrazoxane may interfere with doxorubicin's ability to kill tumor cells (Yeh et al., Circulation 109, 3122-3131, 2004). Furthermore, usage of dexrazoxane also negatively affects bioactivities where iron is normally required (Dorr et al., Semin. Oncol. 23, 23-34, 1996). Thus, it is desirable to develop novel drugs that alleviate doxorubicin-induced heart toxicity while preserving its chemotherapeutic potency.

### SUMMARY OF THE INVENTION

In one aspect, the invention provides a method of reducing anthracycline-induced cardiotoxicity, comprising administering, to a patient receiving an anthracycline, a compound of Formula (I):

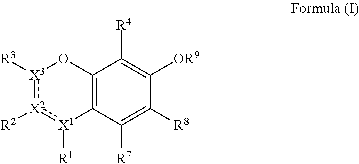

or a pharmacecutically acceptable salt thereof, wherein:
X¹R¹ taken together represent C═O, and X² and X³ together represent C═C; or
X³R³ taken together represent C═O, and X² and X¹ form together C═C;
R⁹ represents H, alkyl, or acyl or, taken together with either R⁴ or R⁸ forms a furan ring substituted with R⁵ and R⁶;
R⁴ and R⁸, when not forming a ring with R⁹, are each independently selected from H, OH, alkyl, acyloxy, and alkoxy;
and the rest of R′, R², R³, R⁵, R⁶ and R⁷ are each independently selected from H, OH, alkyl, acyloxy, and alkoxy.

In certain embodiments of Formula (I), the compound is a compound of Formula (II):

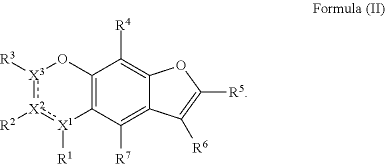

In other embodiments of Formula (I), the compound is a compound of Formula (III):

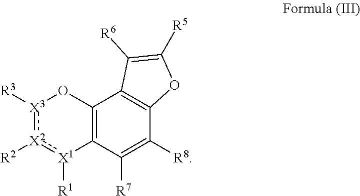

In certain embodiments of Formulas (I)-(III), when X¹R¹ taken together represent C═O, and X² and X³ together represent C═C, then R³ is not methyl and/or R⁷ is not methoxy.

In certain preferred embodiments, at least one of R⁷ and R⁴ is OH, alkoxy, or acyloxy.

In preferred embodiments of Formulas (I)-(III), alkyl, acyloxy, and alkoxy substituents are lower alkyl, lower acyloxy, and lower alkoxy substituents, respectively. In certain such embodiments, R¹, R², R³, R⁴, R⁵, R⁶, R⁷, and R⁸, when not forming a furan ring with R⁹, are each independently selected from hydrogen, methyl, isopropyl, tert-butyl and methoxy. In certain embodiments, the compound is administered after the patient has received the anthracycline.

In certain embodiments, the compound is administered before the patient has received the anthracycline.

In certain embodiments, the compound and the anthracycline are administered simultaneously.

In certain embodiments, the compound reduces anthracycline-induced apoptosis in cardiomyocytes.

In certain embodiments, the compound reduces anthracycline-induced reduction in fractional shortening in cardiomyocytes.

In certain embodiments, the compound reduces anthracycline-induced reduction in strain rate in cardiomyocytes.

In certain embodiments, the compound reduces anthracycline-induced reduction in ejection fraction in cardiomyocytes.

In certain embodiments, the compound the compound does not substantially inhibit anthracycline-induced apoptosis in tumor cells.

In certain embodiments, the anthracycline is selected from daunorubicin, doxorubicin, epirubicin, idarubicin, mitoxantrone, and valrubicin, such as doxorubicin.

In certain embodiments, administering the compound comprises administering a pharmaceutical composition comprising the compound and a pharmaceutically acceptable excipient.

In one aspect, the invention provides a method of reducing anthracycline-induced cardiotoxicity, comprising conjointly administering, to a patient receiving an anthracycline, a compound of Formula (I) and dexrazoxane, diphenyl urea, or a compound of Formula (II):

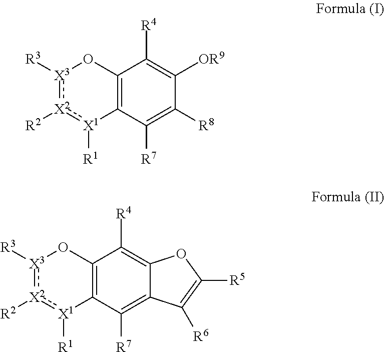

or a pharmacecutically acceptable salt thereof, wherein:
X¹R¹ taken together represent C═O, and X² and X³ together represent C═C; or
X³R³ taken together represent C═O, and X² and X¹ form together C═C; and
R⁹ represents H, alkyl, or acyl or, taken together with either R⁴ or R⁸ forms a furan ring substituted with R⁵ and R⁶;
R⁴ and R⁸, when not forming a ring with R⁹, are each independently selected from H, OH, alkyl, acyloxy, and alkoxy;
the rest of R¹, R², R³, R⁵, R⁶, and R⁷ are each independently selected from H, OH, alkyl, acyloxy, and alkoxy.

In certain embodiments, the invention provides a pharmaceutical composition comprising a compound of Formula (I) and diphenyl urea (DPU), dexrazoxane (DEX) and/or a compound of Formula (II), and a pharmaceutically acceptable excipient, e.g., suitable for use in the above methods. In some such embodiments, the pharmaceutical composition comprises a compound of Formula (I) and dexrazoxane and/or a compound of Formula (II), and a pharmaceutically acceptable excipient.

Exemplary compounds of Formula I include:

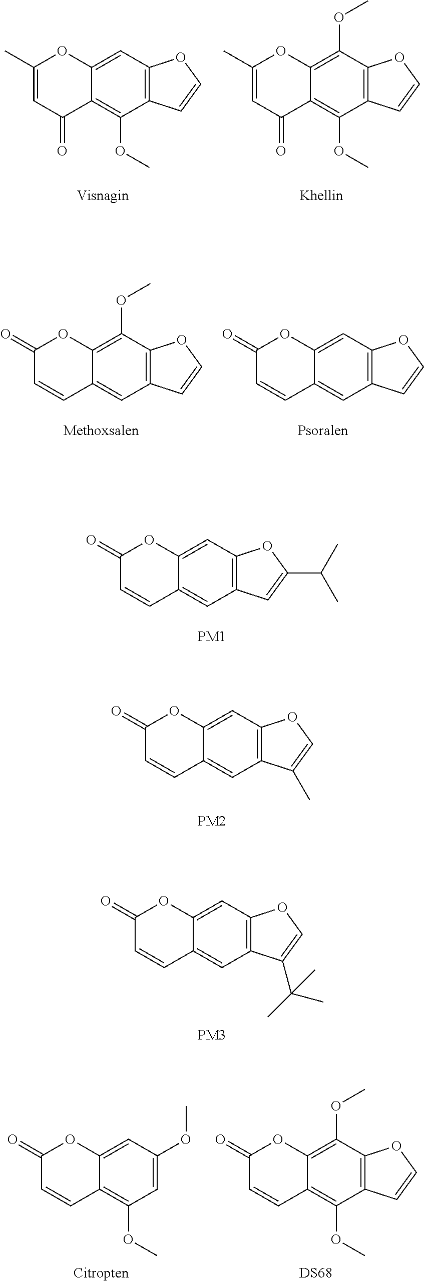

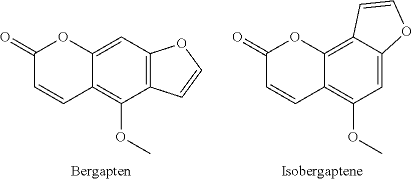 [SMILES: COc1c2ccoc2cc2oc(=O)ccc12 ; COc1cc2occc2c2oc(=O)ccc12 ; "Bergapten" ; "Isobergaptene"]

and their salts (including pharmaceutically acceptable salts).

In one aspect, the invention provides a method of identifying a toxicity-reducing compound comprising treating zebrafish with an anthracycline, adding a test compound, measuring cardiovascular characteristics and/or abnormalities in the treated zebrafish, comparing the cardiovascular characteristics and/or abnormalities of the zebrafish treated with the test compound to cardiovascular characteristics and/or abnormalities of one or more control zebrafish treated with the anthracycline alone.

### BRIEF DESCRIPTION OF THE FIGURES

FIG. 1a shows an experimental scheme for inducing heart failure in zebrafish.

FIG. 1b shows representative images showing normal heart (blue arrow) versus defective heart (red arrow).

FIG. 1c shows conversion of heart contraction using a custom algorithm.

FIG. 1d shows representative images showing numbers of genetically labeled cardiomyocytes.

FIG. 1e shows detection of apoptotic cardiomyocytes by TUNEL staining.

FIG. 1f shows quantification of fractional shortening (FS): (VID_{d}−VIDₛ)/VID_{d}×100.

FIG. 1g shows quantification of cardiomyocyte number.

FIG. 1h depicts quantification of apoptotic cardiomyocytes.

FIGS. 1i and 1j shows visnagin (VIS) and diphenylurea (DPU) rescued heart contraction and blood flow in a dosage-dependent manner.

FIGS. 1k and 1l show representative images indicating that heart morphology is grossly normal in VIS- or DPU-rescued zebrafish.

FIG. 1m shows a representative graph reflecting heart contraction of VIS or DPU rescued zebrafish.

FIG. 1n shows quantification of heart fractional shorting (FS) of DMSO-treated control samples (DMSO), doxorubicin (DOX)-treated samples, doxorubicin and VIS (Dox+VIS) co-treated samples, doxorubicin and DPU (Dox+DPU) co-treated samples. Statistics (compared to doxorubicin treated samples): * p<0.05, ** p<0.01, and *** p<0.001.

FIG. 2a shows quantification of zebrafish heart apoptosis. Labels: DMSO: DMSO-treated control samples; Dox: doxorubicin treated samples; Dox+VIS: doxorubicin and VIS co-treated samples; Dox+DPU: doxorubicin and DPU co-treated samples. Statistics (compared to doxorubicin treated samples): * p<0.05, ** p<0.01, and *** p<0.001.

FIGS. 2b and 2c show quantification of mouse heart apoptosis. Labels: DMSO: DMSO-treated control samples; Dox: doxorubicin treated samples; Dox+VIS: doxorubicin and VIS co-treated samples; Dox+DPU: doxorubicin and DPU co-treated samples. Statistics (compared to doxorubicin treated samples): * p<0.05, ** p<0.01, and *** p<0.001.

FIG. 3a shows that VIS and DPU inhibit doxorubicin-induced apoptosis in neonatal rat cardiomyoyte as measured by TUNEL staining Labels: DM: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin treated samples): * p<0.05, ** p<0.01, and *** p<0.001.

FIG. 3b shows that VIS and DPU inhibit doxorubicin-induced apoptosis in neonatal rat cardiomyoyte as measured by AnnexinV staining Labels: DM: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin treated samples): * p<0.05, ** p<0.01, and *** p<0.001.

FIG. 3c shows that VIS and DPU reduce doxorubicin-induced apoptosis in cardiomyoyte HL1 as measured by AnnexinV staining Labels: DM: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin treated samples): * p<0.05, ** p<0.01, and *** p<0.001.

FIG. 3d shows that VIS and DPU do not reduce doxorubicin-induced apoptosis in tumor line DU145. Labels: DM: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin treated samples): * p<0.05, ** p<0.01, and *** p<0.001.

FIG. 3e shows that VIS and DPU do not reduce doxorubicin-induced apoptosis in tumor line LNCaP. Labels: DM: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin treated samples): * p<0.05, ** p<0.01, and *** p<0.001.

FIG. 3f shows that VIS and DPU do not reduce doxorubicin-induced apoptosis in tumor line MCF7. Labels: DM: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin-treated samples): * p<0.05, ** p<0.01, and *** p<0.001.

FIG. 3g shows that VIS and DPU (20 μM) increase viability of doxorubicin-treated cardiomyocyte HL1. Labels: Dox: doxorubicin-treated samples; Dox+VIS: doxorubicin and VIS co-treated samples; Dox+DPU: doxorubicin and DPU co-treated samples. Statistics (compared to doxorubicin-treated samples): * p<0.05, ** p<0.01, and *** p<0.001.

FIG. 3h shows that VIS and DPU (20 μM) do not increase viability of doxorubicin-treated prostate tumor cell DU145. Labels: Dox: doxorubicin-treated samples; Dox+VIS: doxorubicin and VIS co-treated samples; Dox+DPU: doxorubicin and DPU co-treated samples. Statistics (compared to doxorubicin-treated samples): * p<0.05, ** p<0.01, and *** p<0.001.

FIG. 3i shows that the differential protected could be mediated by selective suppression of caspase activities in cardiac cells. DMSO: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin-treated samples): * p<0.05, ** p<0.01, and *** p<0.001.

FIG. 3j shows that the differential protected could not be mediated by selective suppression of caspase activities in tumor cells. DMSO: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin-treated samples): * p<0.05, ** p<0.01, and *** p<0.001.

FIG. 4a shows that VIS and DPU inhibit doxorubicin-induced JNK phosphorylation in HL 1 cells.

FIG. 4b shows quantification of JNK activation in HL 1 cells. Labels: DMSO: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin treated samples): * p<0.05, ** p<0.01, and *** p<0.001.

FIG. 4c shows that JNK responsive reporter AP-1 luciferase activity is repressed by VIS or DPU in HL 1 cells. Labels: DMSO: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin-treated samples): * p<0.05, ** p<0.01, and *** p<0.001.

FIG. 4d shows that VIS and DPU do not inhibit doxorubicin-induced JNK phosphorylation in DU145 cells.

FIG. 4e shows quantification of JNK activation in DU145 cells. Labels: DMSO: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin-treated samples): * p<0.05, ** p<0.01, and *** p<0.001.

FIG. 4f shows that JNK responsive reporter AP-1 luciferase activity is not repressed by VIS or DPU in DU145 cells. Labels: DMSO: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin treated samples): * p<0.05, ** p<0.01, and *** p<0.001.

FIG. 4g shows that JNK inhibitor SP600125 represses AP-1 luciferase activity in HL 1 cells. Labels: DMSO: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin treated samples): * p<0.05, ** p<0.01, and *** p<0.001.

FIG. 4h shows that JNK inhibitor SP600125 increases HL 1 cell viability. Labels: DMSO: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin-treated samples): * p<0.05, ** p<0.01, and *** p<0.001.

FIG. 4i shows that JNK inhibitor SP600125 represses AP-1 luciferase activity in DU 145 cells. Labels: DMSO: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin-treated samples): * p<0.05, ** p<0.01, and *** p<0.001.

FIG. 4j shows that JNK inhibitor SP600125 increases DU 145 cell viability. Labels: DMSO: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin-treated samples): * p<0.05, ** p<0.01, and *** p<0.001.

FIG. 4 k shows that JNK inhibitor SP600125 reverses deteriorated heart contraction by doxorubicin.

FIG. 4l shows that JNK inhibitor SP600125 reverses deteriorated heart contraction by doxorubicin. Labels: DMSO: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin treated samples): * p<0.05, ** p<0.01, and *** p<0.001.

FIG. 5a shows representative M-mode endocardiogram images: blue line denotes diastolic left ventricle internal dimension (LVIDd) and red line denotes systolic left ventricle internal dimension LVIDs.

FIG. 5b shows that acute treatment with a relatively high dose of doxorubicin reduces fractional shortening in treated mice while VIS partially reversed the reduction. Labels: DMSO: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin treated samples): * p<0.05, ** p<0.01, and *** p<0.001.

FIG. 5c shows that acute treatment with a relatively high dose of doxorubicin reduces strain rate in treated mice while VIS partially reversed the reduction. Labels: DMSO: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin treated samples): * p<0.05, ** p<0.01, and *** p<0.001.

FIG. 5d shows representative M-mode endocardiogram images: blue line denotes diastolic left ventricle internal dimension (LVIDd) and red line denotes systolic left ventricle internal dimension LVIDs.

FIG. 5e shows that chronic treatment with relatively low doses of doxorubicin (5 times over 5 weeks) reduces fractional shortening in treated mice while VIS partially reversed the reduction. Labels: DMSO: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin treated samples): * p<0.05, ** p<0.01, and *** p<0.001.

FIG. 5f shows that chronic treatment with relatively low doses of doxorubicin (5 times over 5 weeks) reduces strain rate in treated mice while VIS partially reversed the reduction. Labels: DMSO: DMSO-treated control samples; Dox: doxorubicin-treated samples; Dox+VIS: doxorubicin and VIS co-treated samples; Dox+DPU: doxorubicin and DPU co-treated samples. Statistics (compared to doxorubicin treated samples): * p<0.05, ** p<0.01, and *** p<0.001.

FIGS. 6a-6e show the effect of various compounds of Formula (I), chromone, coumarin, and benzofuran on the viability of doxorubicin-treated cardiomyocyte HL1. VIS: visnagin; BenzF: benzofuran; Chrom: chromone; Couma: coumarin; Khe: khellin; Meth: methoxsalen; Berg: bergapten; pso: psoralen; Citrop: citropten; Isoberg: isobergaptene.

FIG. 7a shows the effect of combining VIS (at various concentrations) and DPU (20 μM) on the viability of cardiomyocyte HL 1 cells treated with doxorubicin (5 μM).

FIG. 7b shows the effect of combining VIS (at various concentrations) and dexrazoxane (DEX) (100 μM) on the viability of cells treated with doxorubicin (5 μM).

### DETAILED DESCRIPTION OF THE INVENTION

The invention provides methods of reducing anthracycline-induced cardiotoxicity by administering one or more toxicity-reducing compounds, such as a compound of Formula (I), to a patient receiving an anthracycline. In particular, the invention provides methods of reducing doxorubicin-induced toxicity by administering one or more toxicity-reducing compounds, such as a compound of Formula (I), to a patient receiving doxorubicin. “Patients receiving an anthracycline”, as the term is used herein, include patients who have been administered at least one dose of an anthracycline within the prior week, patients who are prescribed to receive at least one dose of an anthracycline within a week after receiving a toxicity-reducing compound, and patients who are otherwise being conjointly treated with an anthracycline and toxicity-reducing compound. The invention also provides methods of identifying a toxicity-reducing compound.

In certain embodiments, the toxicity-reducing compound is a compound of Formula (I), (II), or (III) or a pharmaceutically acceptable salt thereof. In other embodiments, the toxicity-reducing compound is diphenylurea.

Another aspect of the invention provides a method of administering an anthracycline to a patient, comprising conjointly administering at least one toxicity-reducing compound with the anthracycline. Such conjoint administration may be achieved by way of the simultaneous, sequential or separate dosing of the individual components of the administration.

In certain embodiments, the toxicity-reducing compound is administered before the anthracycline. For example, the toxicity-reducing compound may be administered at least 1 minute before the anthracycline, at least 5 minutes before the anthracycline, at least 15 minutes before the anthracycline, at least 30 minutes before the anthracycline, or even at least 60 minutes before the anthracycline.

In certain other embodiments, the toxicity-reducing compound is administered after the anthracycline. For example, the toxicity-reducing compound may be administered at least 1 minute after the anthracycline, at least 5 minutes after the anthracycline, at least 15 minutes after the anthracycline, at least 30 minutes after the anthracycline, or even at least 60 minutes after the anthracycline.

In other embodiments, the toxicity-reducing compound is administered simultaneously with the anthracycline, such as in a single co-formulation with the anthracycline.

Another aspect of the invention provides a kit for reducing the toxicity induced by anthracyclines. In certain such embodiments, the kit contains a toxicity-reducing compound, such as a compound of Formula (I), (II), or (III), and instructions for administering the toxicity-reducing agent with an anthracycline. The kit may optionally further include an anthracycline. The toxicity-reducing compound and/or the anthracycline (if present) may be provided as pharmaceutical preparations, whether for administration by the same route of administration (e.g., intravenous), or by differing routes of administration (e.g., the anthracycline in an intravenous formulation and the toxicity-reducing compound as an oral formulation). The kit may include one or more toxicity-reducing compounds, which may be formulated separately or together.

In some embodiments, the toxicity-reducing compound has cardioprotective properties.

In some embodiments, the cardioprotective properties of the compound can be characterized by the reduction of anthracycline-induced apoptosis in cardiomyocytes.

In some embodiments, the cardioprotective properties of the compound can be characterized by the reduction of anthracycline-induced fractional shortening in cardiomyocytes.

In some embodiments, the cardioprotective properties of the compound can be characterized by the reduction of anthracycline-induced in strain rate in cardiomyocytes.

In some embodiments, the cardioprotective properties of the compound can be characterized by the reduction of anthracycline-induced in ejection fraction in cardiomyocytes.

### I. DEFINITIONS

The term “alkoxy” refers to an oxygen having an alkyl group attached thereto. Representative alkoxy groups include methoxy, ethoxy, propoxy, tert-butoxy and the like.

The term “acyloxy” refers to means a straight-chain or branched alkanoyl group having 1 to 6 carbon atoms, such as formyl, acetyl, propanoyl, butyryl, valeryl, pivaloyl and hexanoyl, and arylcarbonyl group described below, or a heteroarylcarbonyl group described below. The aryl moiety of the arylcarbonyl group means a group having 6 to 16 carbon atoms such as phenyl, biphenyl, naphthyl, or pyrenyl. The heteroaryl moiety of the heteroarylcarbonyl group contains at least one hetero atom from O, N, and S, such as pyridyl, pyrimidyl, pyrroleyl, furyl, benzofuryl, thienyl, benzothienyl, imidazolyl, triazolyl, quinolyl, iso-quinolyl, benzoimidazolyl, thiazolyl, benzothiazolyl, oxazolyl, and indolyl.

The term “alkyl” refers to the radical of saturated aliphatic groups, including straight-chain alkyl groups, and branched-chain alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. In certain embodiments, alkyl groups are lower alkyl groups, e.g. methyl, ethyl, n-propyl, i-propyl, n-butyl and n-pentyl.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. In certain embodiments, a straight chain or branched chain alkyl has 30 or fewer carbon atoms in its backbone (e.g., C₁-C₃₀ for straight chains, C₃-C₃₀ for branched chains). In preferred embodiments, the chain has ten or fewer carbon (C₁-C₁₀) atoms in its backbone. In other embodiments, the chain has six or fewer carbon (C₁-C₆) atoms in its backbone.

Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, an alkylthio, an acyloxy, a phosphoryl, a phosphate, a phosphonate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aryl or heteroaryl moiety.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. Examples of straight chain or branched chain lower alkyl include methyl, ethyl, isopropyl, propyl, butyl, tertiary-butyl, and the like.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of the invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, an alkylthio, an acyloxy, a phosphoryl, a phosphate, a phosphonate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety.

Unless specifically stated as “unsubstituted,” references to chemical moieties herein are understood to include substituted variants. For example, reference to an “alkyl” group or moiety implicitly includes both substituted and unsubstituted variants.

At various places in the present specification substituents of compounds of the invention are disclosed in groups or in ranges. It is specifically intended that the invention include each and every individual subcombination of the members of such groups and ranges. For example, the term “C₁-C₆ alkyl” is specifically intended to individually disclose methyl, ethyl, propyl, isopropyl, n-butyl, sec-butyl, isobutyl, etc.

For a number qualified by the term “about”, a variance of 2%, 5%, 10% or even 20% is within the ambit of the qualified number

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, the term “treating” or “treatment” includes reversing, reducing, or arresting the symptoms, clinical signs, and underlying pathology of a condition in manner to improve or stabilize a subject's condition. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “anthracycline” refers to a class of antineoplastic antibiotics having an anthracenedione (also termed anthraquinone or dioxoanthracene) structural unit. For example, the term “anthracycline” is specifically intended to individually include daunorubicin, doxorubicin, epirubicin, idarubicin, valrubicin, ditrisarubicins, mitoxantrone, etc.

The phrase “conjoint administration” refers to any form of administration in combination of two or more different therapeutic compounds such that the second compound is administered while the previously administered therapeutic compound is still effective in the body (e.g., the two compounds are simultaneously effective in the patient). For example, the different therapeutic compounds can be administered either in the same formulation or in separate formulations, either concomitantly or sequentially.

### II. PHARMACEUTICAL COMPOSITIONS

Toxicity-reducing compounds may be provided in a pharmaceutical composition, e.g., combined with a pharmaceutically acceptable carrier, for administration to a patient. Such a composition may also contain diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. Such additional factors and/or agents may be included in the pharmaceutical composition to produce a synergistic effect with, or minimize the side effects of the toxicity-reducing compounds.

The pharmaceutical compositions may be in the form of a liposome or micelles in which the the toxicity-reducing compounds are combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

The terms “pharmaceutically effective amount” or “therapeutically effective amount”, as used herein, means the total amount of each active component of the pharmaceutical composition or method that is sufficient to show a meaningful patient benefit, e.g., treatment, healing, prevention, inhibition or amelioration of a physiological response or condition, such as an inflammatory condition or pain, or an increase in rate of treatment, healing, prevention, inhibition or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

Each of the methods or uses of the present invention, as described herein, comprises administering to a mammal in need of such treatment or use a pharmaceutically or therapeutically effective amount of a toxicity-reducing compound, or a pharmaceutically acceptable salt or ester form thereof. Compounds may be administered alone or in combination with the anthracycline and/or other medications.

Administration of toxicity-reducing compounds used in the pharmaceutical composition or to practice the methods of the present invention can be carried out in a variety of conventional ways, such as oral ingestion, inhalation, or cutaneous, subcutaneous, or intravenous, intramuscular, and intraperitoneal injection.

When a therapeutically effective amount of a toxicity-reducing compound(s) is administered orally, the compound(s) of the present invention may be in the form of a tablet, capsule, powder, solution or elixir. When administered in tablet form, the pharmaceutical composition may additionally contain a solid carrier such as a gelatin or an adjuvant. The tablet, capsule, and powder may contain from about 5 to 95% of a toxicity-reducing compound, and preferably from about 10% to 90% of a toxicity-reducing compound. When administered in liquid form, a liquid carrier such as water, petroleum, oils of animal or plant origin such as peanut oil, mineral oils, phospholipids, tweens, triglycerides, including medium chain triglycerides, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition typically contains from about 0.5 to 90% by weight of a toxicity-reducing compound, and preferably from about 1 to 50% by weight of a toxicity-reducing compound.

When a therapeutically effective amount of a toxicity-reducing compound(s) is administered by intravenous, cutaneous or subcutaneous injection, such compound(s) may be in the form of a pyrogen-free, parenterally acceptable aqueous solution. The preparation of such parenterally acceptable solutions, having due regard to pH, isotonicity, stability, and the like, is within the skill in the art. A preferred pharmaceutical composition for intravenous, cutaneous, or subcutaneous injection should contain, in addition to the toxicity-reducing compounds, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the toxicity-reducing compound may also contain stabilizers, preservatives, buffers, antioxidants, or other additives known to those of skill in the art.

The amount of the toxicity-reducing compound(s) in the pharmaceutical composition will depend upon the nature and severity of the cardiotoxicity, on the amount of the anthracycline used, and on the nature of prior treatments the patient has undergone. Ultimately, the practitioner will decide the amount of the toxicity-reducing compound with which to treat each individual patient. Initially, the practitioner may administer low doses of the toxicity-reducing compound and observe the patient's response. Larger doses of compounds of the toxicity-reducing compound may be administered until the optimal therapeutic effect is obtained for the patient, and at that point the dosage is not increased further. Representative doses of the present invention include, but are not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg and about 0.001 mg to about 25 mg. Multiple doses may be administered during one day, especially when relatively large amounts are deemed to be needed. It is contemplated that the various pharmaceutical compositions used to practice the methods of the present invention should contain about 0.1 μg to about 100 mg (preferably about 0.1 mg to about 50 mg, more preferably about 1 mg to about 2 mg) of toxicity-reducing compound per kg body weight.

The duration of intravenous therapy using the pharmaceutical composition of the toxicity-reducing compound will vary, depending on the severity of the cardiotoxicity, the amount of the anthracycline used and potential idiosyncratic response of each individual patient. It is contemplated that the duration of each application of the toxicity-reducing compounds will be in the range of 12 to 24 hours of continuous intravenous administration. Ultimately the practitioner will decide on the appropriate duration of intravenous therapy using the pharmaceutical composition of the toxicity-reducing compounds.

The use of liposomal preparations of anthracyclines, in particular doxorubicin, can be used to reduce the risk of cardiotoxicity induced by anthracyclines. Representative liposomal preparations are disclosed by Mayer et al. in US 2012/0009252, the teachings of which are incorporated by reference herein in their entirety as they relate to liposomal preparations of anthracyclines. In some embodiments, the methods of the invention comprise administering an anthracycline, such as doxorubicin, as a liposomal formulation.

### III. EXEMPLIFICATION

The invention now being generally described, it will be more readily understood by reference to the following examples which are included merely for purposes of illustration of certain aspects and embodiments of the present invention, and are not intended to limit the invention.

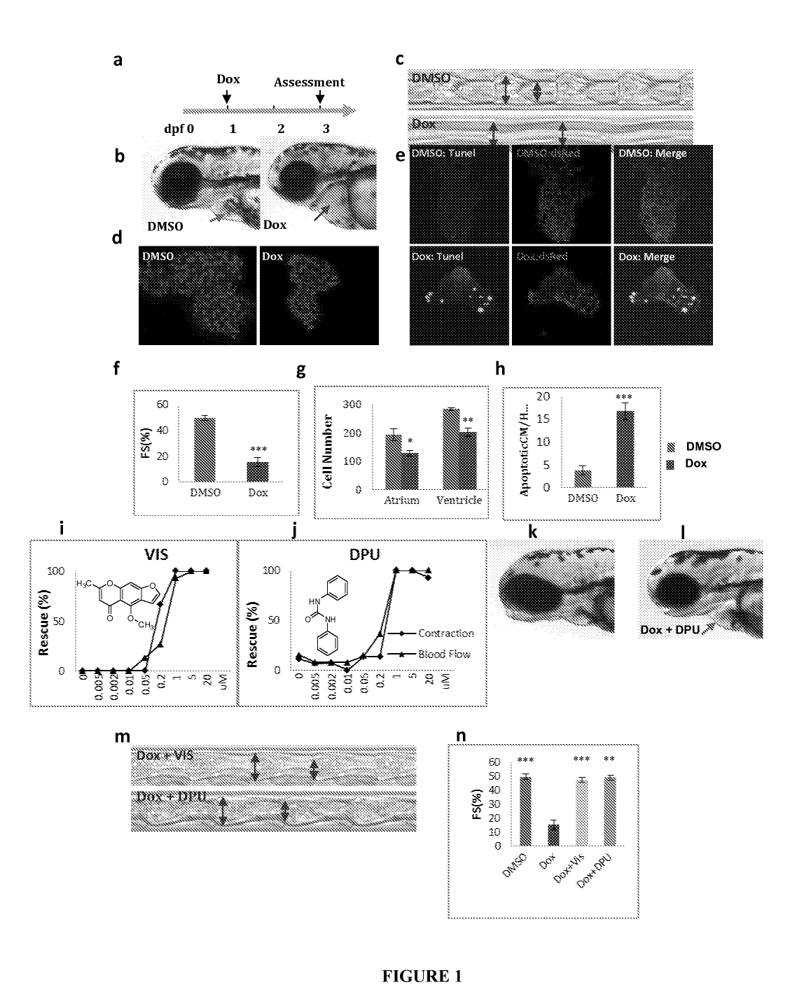

Several compounds of Formula (I), including visnagin (VIS), and diphenylurea (DPU), were identified as preventing doxorubicin-induced decreases in cardiac contraction and circulation. VIS and DPU were protective at concentrations below 1 μM (FIGS. 1i and 1j) and prevented the overt morphological effects of doxorubicin on the heart, including ventricular compaction and pericardial edema (FIGS. 1k and 1l). Both compounds also completely rescued cardiac contractility, as measured by fractional shortening (FIGS. 1m and 1n). Therefore, VIS and DPU represent two classes of compounds that potently protects the heart from the toxic effects of anthracyclines, in particular doxorubicin.

### Example 1

### Zebrafish Model

Cmlc2-EGFP fish larvae 28 (Rottbauer et al., Circ. Res. 99, 323-331, 2006) (dpf 1) were arrayed into 96-well plates, with each well containing three fish in 200 μM E3 buffer with 100 pM doxorubicin. For screening, about 400 nl of small molecule stock solution was transferred from 96-well format library plates to fish plate with transfer pins. At dpf 3, treated fish were screened under inverted fluorescent microscope (100×) for heart contraction and tail circulation.

### Example 2

### Doxorubicin-induced Heart Failure (HF) Model in Zebrafish

Zebrafish have been used successfully for high-throughput screening (HTS) to identify chemical compounds that suppress genetic defects and other disease states (Peterson et al., Methods Cell. Biol. 105, 525-541, 2011). Compared to cell-based in vitro systems, in vivo screening offers several advantages, including the ability to discover compounds with therapeutic activity even without knowing their molecular targets. In addition, compounds discovered by in vivo screening are selected for their ability to be effective in the complex context of the disease of interest. Therefore a zebrafish model of doxorubicin-induced heart failure was established to identify compounds that protect the heart from doxorubicin. To avoid interference with the early cardiogenic process, we started to treat zebrafish one day post-fertilization (dpf) with 100 μM doxorubicin and assessed phenotypic changes at 3 dpf (FIG. 1a). Two days after being exposed to doxorubicin, fish exhibited extensive pericardial edema. Microscopic examination revealed that the heart atrium was collapsed and the ventricle elongated (FIG. 1b). Heart contraction was dramatically compromised, resulting in the absence of blood cell circulation within tail blood vessels. Using a high-speed camera and a custom analysis algorithm, we were able to calculate the fractional shortening of the zebrafish hearts. The results demonstrated that both heart rate and contractility were dramatically reduced in doxorubicin-treated fish (FIGS. 1 c and 1f). Using a cmcl2-dsRed transgenic line, we counted cardiomyocyte number and found that both atrium and ventricle cardiomyocyte numbers were significantly reduced compared to controls (FIG. 1d and 1g). TUNEL staining also showed that doxorubicin increased cardiomyocyte apoptosis (FIG. 1e and 1h). Therefore, the zebrafish model appears to recapitulate several key aspects of the doxorubicin-induced heart failure seen in humans, including increased apoptosis and reduced contractility. Because the doxorubicin-induced changes in cardiac function and blood circulation are easily detected visually, they formed the basis of an assay to identify small molecules suppressing doxorubicin-induced cardiotoxicity.

Cmlc2-nuc-dsRed fish (dpf 1) were treated with DMSO or 100 μM doxorubicin for two days. Hearts were surgically removed and briefly fixed with 4% paraformaldehyde (PFA) for 20 minutes at room temperature. The fixed heart samples were embedded in mounting medium and flattened with slide and coverslip. Confocal images were captured from the flattened hearts, and red nuclei were counted. All experiments were done at least three times, and the statistics were obtained by Student T-test.

### Example 3

### VIS and DPU Reduce Doxorubicin-Induced Apoptosis in Zebrafish

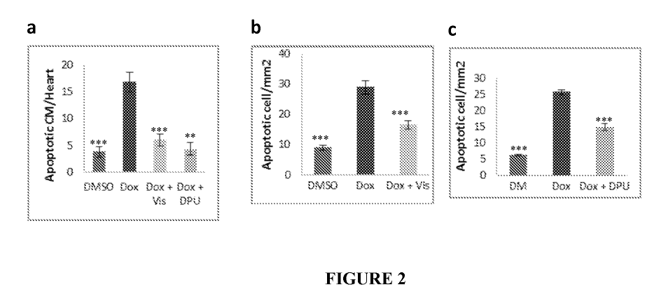

Because cardiomyocyte apoptosis plays a critical role in mediating doxorubicin-induced cardiomyopathy, we sought to determine if VIS Aand DPU attenuate heart failure by inhibiting cardiomyocyte apoptosis. We used a transgenic zebrafish line expressing nuclear DsRed from the cmlc2 promoter to mark cardiomyocytes and performed TUNEL staining to identify apoptotic cells after treatment with doxorubicin (Supplement FIG. 1a). As shown in FIG. 2a, doxorubicin treatment caused a four-fold increase in apoptotic cardiomyocytes in 4 dpf zebrafish. VIS and DPU greatly reduced the number of apoptotic cardiomyocytes induced by doxorubicin, nearly restoring the level of apoptosis to the level of controls.

Cmlc2-nuc-dsRed fish larvae (dpf 1) were treated with DMSO, 100 μM doxorubicin or doxorubicin plus 20 μM rescue compounds for two days. Hearts were surgically removed and fixed with 4% paraformaldehyde (PFA) for 20 minutes at room temperature. After staining with an in situ TUNEL kit (Roche), heart samples were embedded in mounting medium and flattened with slide and coverslip, and then subjected to confocal microscopy. The data were quantified as percentage of TUNEL positive cardiomyocytes. All experiments were done at least three times, and the statistics were obtained by Student T-test.

### Example 4

### VIS and DPU Reduce Doxorubicin-Induced Apoptosis in Mice

To determine if the ability of VIS and DPU to protect cardiomyocytes from apoptosis was conserved in mammals, we treated mice with doxorubicin with or without co-treatment with VIS or DPU. One day later, hearts were collected for apoptosis assays using TUNEL (Supplement FIG. 1b). As seen in zebrafish, doxorubicin caused a 3- to 4-fold increase in apoptosis in mouse cardiac sections. Co-treatment with VIS or DPU significantly decreased doxorubicin-induced apoptosis (FIGS. 2b and 2c). Therefore, VIS and DPU appear to prevent doxorubicin-induced cardiac apoptosis in both zebrafish and mice.

Male C57BL/6 mice between 8-10 weeks were injected with DMSO, 15 mg/kg doxorubicin, or doxorubicin plus 25 mg/kg visnagin, or doxorubicin plus 10 mg/kg diphenylurea intraperitoneally. One day after injection, hearts were collected and fixed with 4% PFA at 4° C. overnight and then were subjected to paraffin embedding and sectioning. The heart sections were stained using the TMR TUNEL kit (Roche) following the manufacturer's protocol. The stained sections were scanned with MetaMorph-assisted slide scanning microscopy, and apoptosis indices were quantified as TUNEL positive cell/mm². All experiments were done at least three times, and the statistics were obtained by Student T-test.

### Example 5

### Cultured Cells

Cardiomyocyte line HL-1 was derived from mouse atrium tumor. Culturing conditions were previously published (Claycomb et al., Proc. Natl. Acad. Sci. USA, 95, 2979, 1998). HL 1 was cultured with Claycomb medium (Sigma) supplemented with fetal bovine serum (FBS), penicillin/streptomycin, L-glutamine and norepinephrine. DU145 and LNCaP are human prostate tumor lines, while MCF7 is a human breast cancer line. DU145 and MCF7 were cultured with high glucose DMEM (Life Sciences) supplemented with penicillin/streptomycin and L-glutamine, and LNCaP was grown in RPMI-1640 (Life Sciences) plus penicillin/streptomycin and L-glutamine.

### Example 6

### VIS Selectively Protects Cardiac but not Tumor Cells

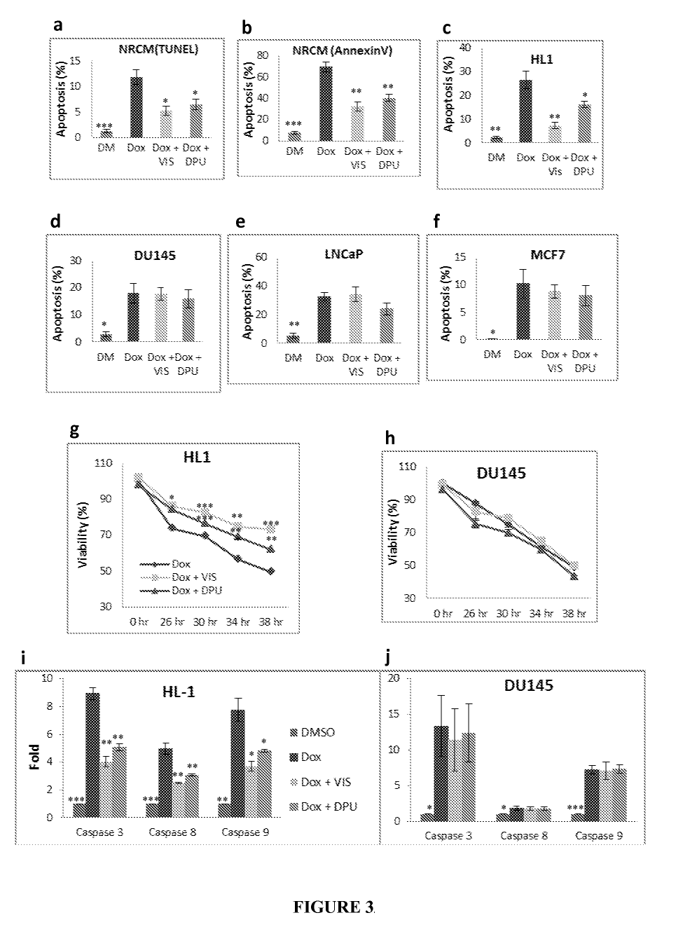

To determine whether VIS or DPU could protect tumor cells from doxorubicin-mediated killing, we tested multiple tumor cell lines including two prostate tumor lines DU145 and LNCaP, and a breast cancer line MCF7. The results showed that VIS or DPU consistently improved the viability of cardiomyocytes but not tumor cells (FIGS. 3a and 3b; supplement FIGS. 2a, 2b, 2 c and 2d).

Cultured HL1 and DU145 cells were treated with doxorubicin for the indicated amounts of time and were assayed using CellTiter Glo Luminescent Cell Viability Assay (Promega). All experiments were done at least three times, and the statistics were obtained by Student T-test.

Cultured HL1, DU145, LNCaP and MCF7 cells were stained with fluorescent Annexin V (Roche) and were live imaged after being treated with doxorubicin for the indicated amounts time. Apoptosis indices were quantified as percentage of AnnexinV positive cells. All experiments were done at least three times, and the statistics were obtained by Student T-test.

Doxorubicin treatment activated caspases 3, 8 and 9, representing common, extrinsic, and intrinsic apoptosis pathways respectively, in both cardiac and tumor cells. However, the activity of the three caspases was inhibited by VIS or DPU in cardiomyocyte but not tumor cells (FIGS. 3c and 3d). Without being bound by a specific theory, this result suggests that VIS or DPU do not function as general caspase inhibitors but rather inhibit caspase activity in a cell type-specific manner. We also examined effects of the compounds on apoptosis (via TUNEL staining) in cardiomyocytes and tumor cells. Consistent with their effects on caspase activity, doxorubicin increased apoptosis in all treated cells, while VIS or DPU specifically suppressed apoptosis in cardiomyocytes but not tumor cells (FIGS. 3e, 3f, 3g and 3h; Supplemental FIGS. 2e, 2f, 2 g and 2h).

Specific activity of caspases 3, 8 and 9 were assessed semi-quantitatively using the corresponding fluorogenic substrates following manufacturer's protocols (R&D Systems). All experiments were done at least three times, and the statistics were obtained by Student T-test.

### Example 7

### VIS Selectively Inhibits Doxorubicin-Induced JNK Phosphorylation in HL 1Cells

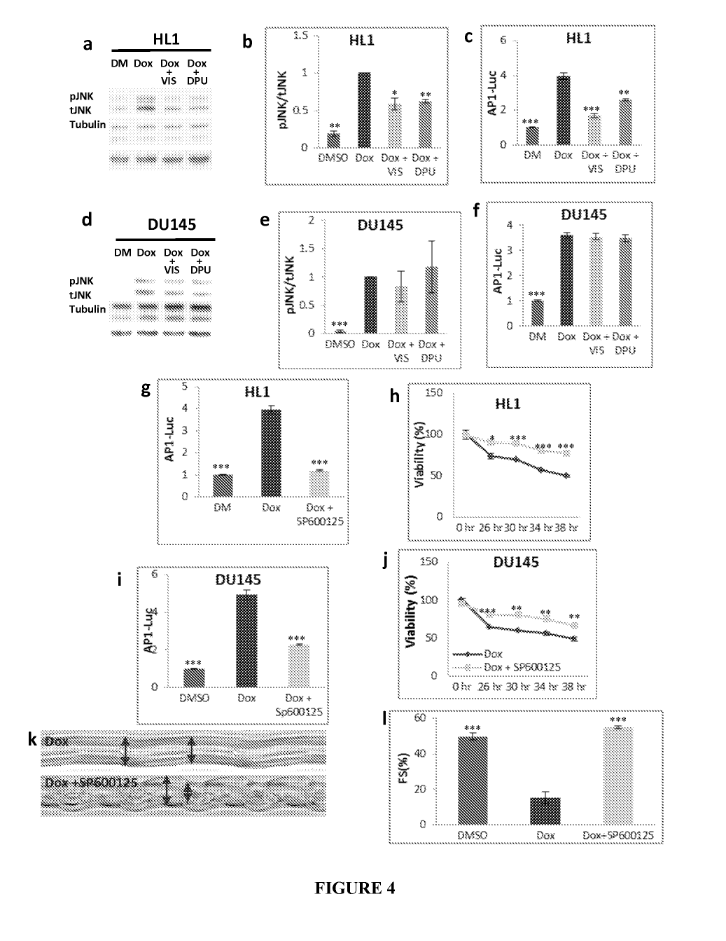

In an effort to identify pathways involved in VIS and DPU mediated cardioprotection, we screened a panel of kinases by western blotting. We found that JNK was activated by doxorubicin in both cardiomyocyte-derived cell line (HL 1) and prostate tumor-derived line DU145 (FIGS. 4a and 4d). Of interest, JNK activation was selectively suppressed by VIS treatment only in HL 1 but not in DU145 cells (FIGS. 4a, 4b, 4d and 4e). To confirm the cardiomyocyte-specific inhibition of JNK signaling, we used a luciferase-based AP1 reporter assay to detect JNK activation in HL 1 and DU145 cells. Again, doxorubicin induced AP1 reporter activity in both cell types, but VIS and DPU suppressed the activation only in the HL 1 cells (FIGS. 4c and 4f). To test further the idea that JNK inhibition could underlie the ability of VIS to promote doxorubicin resistance, we employed a JNK specific inhibitor SP60012516. At concentrations where SP600125 overtly inhibited AP1 luciferase reporter activity (FIGS. 4g and 4i), it significantly improved survival of both HL 1 and DU145 cells exposed to doxorubicin (FIGS. 4h and 4j). Furthermore, SP600125 completely reversed the cardiac contractility deficits in doxorubicin treated zebrafish (FIGS. 4k and 4l). Taken together, these findings suggest that VIS may preferentially protect cardiomyocytes via cell type-specific inhibition of JNK signaling.

HL 1 cells treated with DMSO, doxorubicin or doxorubicin plus rescue compounds were harvested with RIPA buffer and then subjected to PAGE, followed by blotting with antiJNK, anti-phosph-JNK (Cell Signaling) or anti-tubulin (Millipore) antibodies. Blots were quantified by densitometry with Image J. All experiments were done at least three times, and the statistics were obtained by Student T-test.

### Example 8

### VIS Improves Cardiac Function in Doxorubicin-Treated Mice

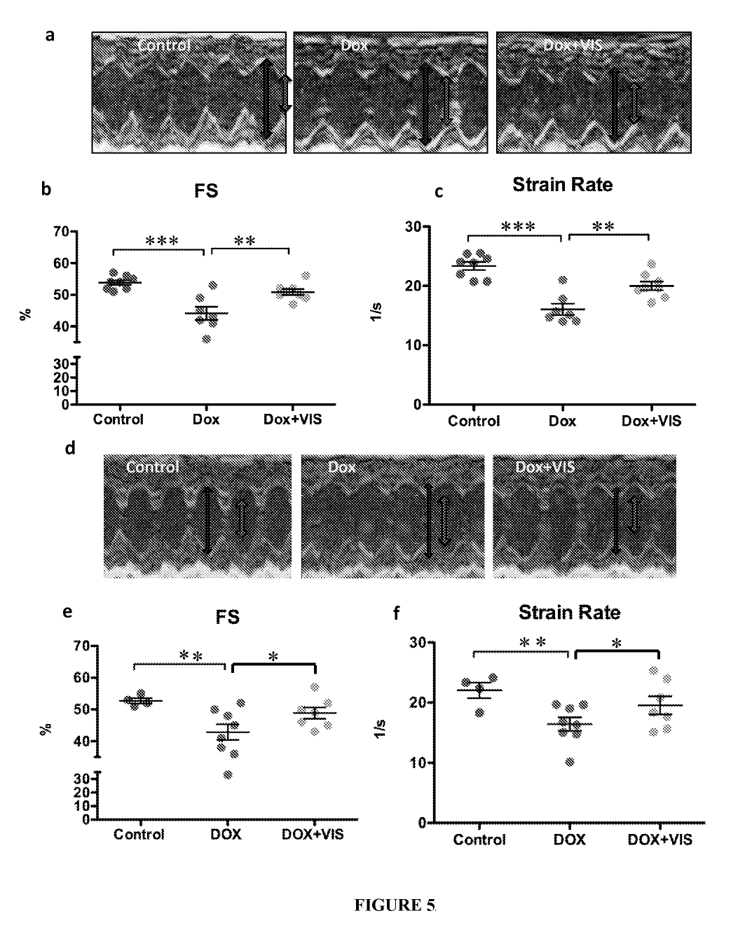

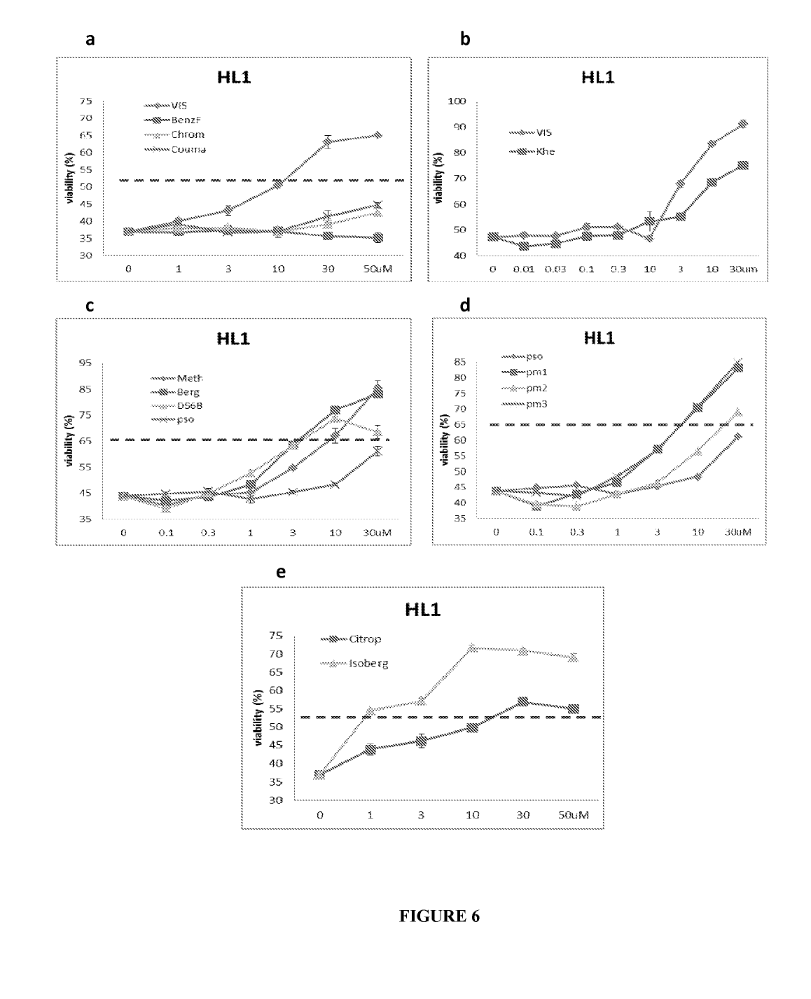

In short term experiments, we showed that VIS significantly decreased doxorubicin induced apoptosis in mouse hearts (FIG. 2). To test whether the reduced apoptosis could be beneficial at a functional level, we employed a doxorubicin heart failure model in which we induced heart failure by injecting 15 mg/kg doxorubicin intraperitoneally. In the treatment group, we injected mice with 25 mg/kg VIS immediately prior to doxorubicin injection. Five days after the initial injection, we assessed heart function by M mode echocardiogram. We found that mice treated with doxorubicin and VIS exhibited significant improvement in both fractional shortening and strain rate compared to mice treated with doxorubicin alone (Table 1, FIG. 5). Therefore, VIS treatment provides a functional benefit to doxorubicin-treated mice.

TABLE 1
Acute Doxorubicin-induced HF Model

| | Control | Dox | Dox + VIS |
|---|---|---|---|
| HR, beats/min | 710 ± 15 | 609 ± 23** | 640 ± 17* |
| LVIDd, mm | 3.22 ± 0.05 | 3.19 ± 0.09 | 3.08 ± 0.06 |
| LVIDs, mm | 1.48 ± 0.04 | 1.78 ± 0.10* | 1.51 ± 0.04‡ |
| FS, % | 54 ± 1 | 44 ± 2*** | 51 ± 1‡‡ |
| Strain Rate, 1/sec | 23 ± 1 | 16 ± 1*** | 20 ± 1*‡‡ |
| N | 8 | 7 | 8 |

Values are presented as mean ± SEM;
*P < 0.05
**P < 0.01
***P < 0.001 vs. Control;
‡P < 0.05,
‡‡P < 0.01, vs. Dox

Strain C57BL/6 male mice between 8-10 weeks of age were purchased from Charles River Laboratories. Acute heart failure was induced by a single intraperitoneal (IP) injection of 15 mg/kg doxorubicin dissolved in saline. In the treatment group, 25 mg/kg visnagin was dissolved in vehicle consisting of 10% ethanol and 90% olive oil, and then injected IP immediately followed by doxorubicin injection. In the control group, plain vehicle and saline were injected instead. Five days later, injected mice were subjected to echocardiography. Five days after treatment, transthoracic echocardiographic images were obtained and interpreted by an echocardiographer blinded to the experimental design using a 13.0-MHz linear probe (Vivid 7; GE Medical System, Milwaukee, Wis.) as described (Zou et al., Crit. Care Med. 38, 1335-1342, 2010; Neilan et al., Eur. Heart J. 27, 1868-1875, 2006). Mice were lightly anesthetized with ketamine (20 mg/kg). M-mode images were obtained from a parasternal short-axis view at the midventricular level with a clear view of papillary muscle. Tissue Doppler imaging was collected at a frame rate of 483 frames per second and a depth of 1 em. LV end-diastolic internal diameter (LVIDd) and LV end systolic internal diameter (LVIDS) were measured. Fractional shortening (FS) was defined as [(LVIDd−LVIDS)/LVIDd]. Strain rate of the posterior wall was analyzed offline in an EchoPAC workstation (GE Healthcare, Wauwatosa, Wis.). A region of interest (axial distance, 0.2 mm; width, 0.6 mm) was manually positioned in the middle of the posterior wall. A strain length of 0.5 mm was used. Peak systolic strain rate was measured. The temporal smoothing filters were turned off for all measurements. The values of three consecutive cardiac cycles were averaged.

### Example 9

### Zebrafish Heart Fractional Shortening Measurement

In order to confirm and quantify the activity of compounds with cardioprotective properties identified in the preceding assays, a high-resolution assay of cardiac function, described by Shin et al. (Physiol Genomics. 2010 July; 42(2): 300-309), can be used to measure heart fractional shortening.

### Example 10

### Combined Effect of VIS and DPU or VIS and Dexrazoxane (DEX) on Cardiomyocytes

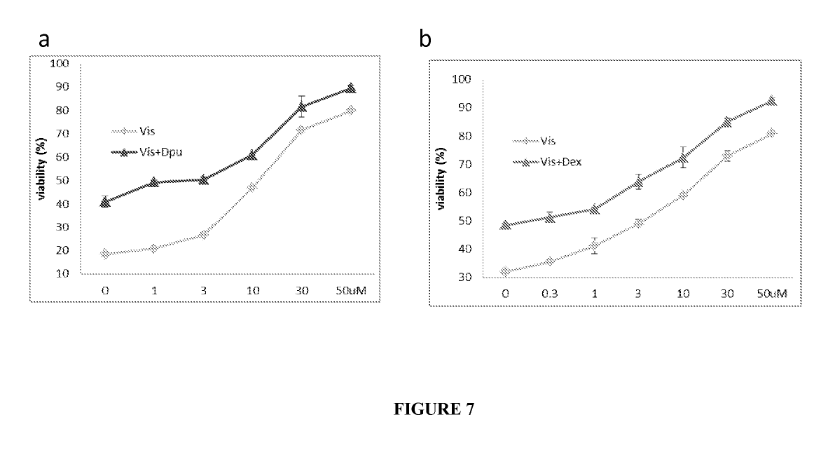

To determine the combined effects of the combinations of VIS/DPU and VIS/dexrazoxane (DEX) in the protection of cardiomyocytes against doxorubicin-mediated killing, we compared the effects of the respective combinations against VIS alone in cardiomyocyte cells HL 1. The results showed that the combinations of VIS/DPU and VIS/DEX improved the viability of cardiomyocytes contacted with an anthracycline compared to treatment of such cells with VIS alone (FIGS. 7a and 7b).

Cultured HL1 cells were treated with doxorubicin followed by treatment with the respective agent or combinations. The cells were harvested twenty-seven hours after treatment and assayed using CellTiter Glo Luminescent Cell Viability Assay (Promega).

All publications and patents cited herein are hereby incorporated by reference in their entirety.

Those skilled in the art will recognize, or be able to ascertain using no more than routine experimentation, many equivalents to the specific embodiments of the invention described herein. Such equivalents are intended to be encompassed by the following claims.

1. A method of reducing anthracycline-induced cardiotoxicity, comprising administering, to a patient receiving an anthracycline, a compound of Formula (I): 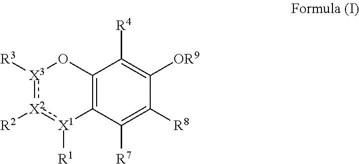
or a pharmacecutically acceptable salt thereof, wherein:
X¹R¹ taken together represent C═O, and X² and X³ together represent C═C; or
X³R³ taken together represent C═O, and X² and X¹ form together C═C; and
R⁹ represents H, alkyl, or acyl or, taken together with either R⁴ or R⁸ forms a furan ring substituted with R⁵ and R⁶;
R⁴ and R⁸, when not forming a ring with R⁹, are each independently selected from H, OH, alkyl, acyloxy, and alkoxy;
the rest of R¹, R², R³, R⁵, R⁶, and R⁷ are each independently selected from H, OH, alkyl, acyloxy, and alkoxy.

2. The method of claim 1, wherein the compound is a compound of Formula (II):

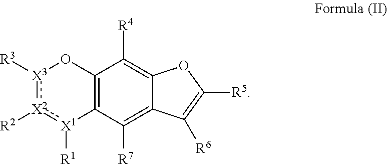

3. The method of claim 1, wherein the compound is a compound of Formula (III):

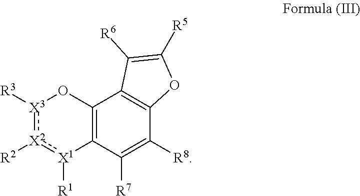

4. The method according to any preceding claim, wherein R¹, R², R³, R⁴, R⁵, R⁶, R⁷, and R⁸, when not forming a furan ring with R⁹, are each independently selected from hydrogen, lower alkyl, lower alkoxy, and lower acyloxy, such as from hydrogen, methyl, isopropyl, tert-butyl and methoxy.

5. The method according to any preceding claim, wherein when X¹R¹ taken together represent C═O, and X² and X³ together represent C═C, then R³ is not methyl and/or R⁷ is not methoxy.

6. The method according to any preceding claim, wherein at least one of R⁷ and R⁴ is OH, alkoxy, or acyloxy.

7. The method according to any preceding claim, wherein the compound is administered after the patient has received the anthracycline.

8. The method according to any one of claims 1-6, wherein the compound is administered before the patient receives the anthracycline.

9. The method according to any one of claims 1-6, wherein the compound and the anthracycline are administered simultaneously.

10. The method according to any preceding claim, wherein the compound reduces anthracycline-induced apoptosis in cardiomyocytes.

11. The method according to any preceding claim, wherein the compound reduces anthracycline-induced reduction in fractional shortening in cardiomyocytes.

12. The method according to any preceding claim, wherein the compound reduces anthracycline-induced reduction in strain rate in cardiomyocytes.

13. The method according to any preceding claim, wherein the compound reduces anthracycline-induced reduction in ejection fraction in cardiomyocytes.

14. The method according to any preceding claim, wherein the compound does not substantially inhibit anthracycline-induced apoptosis in tumor cells.

15. The method according to any preceding claim, wherein the anthracycline is selected from daunorubicin, doxorubicin, epirubicin, idarubicin, mitoxantrone, and valrubicin.

16. The method according to claim 15, wherein the anthracycline is doxorubicin.

17. The method according to any preceding claim, wherein administering the compound comprises administering a pharmaceutical composition comprising the compound and a pharmaceutically acceptable excipient.

18. A method of identifying a toxicity-reducing compound comprising:
treating zebrafish with an anthracycline;
treating the zebrafish with a test compound;
measuring cardiovascular characteristics and/or abnormalities in the treated zebrafish; and
comparing the cardiovascular characteristics and/or abnormalities of the zebrafish treated with the test compound to cardiovascular characteristics and/or abnormalities of one or more control zebrafish treated with the anthracycline alone.
